(19) United States
(12) Patent Application Publication
(10) Pub. No.: US 2008/0188840 A1
(43) Pub. Date: Aug. 7, 2008

(54) HANDPIECE USED FOR COSMETIC OR DERMATOLOGIC TREATMENT

(76) Inventors: Charles Johnson, Northboro, MA (US); Michael Clancy, Westford, MA (US)

Correspondence Address: PROSKAUER ROSE LLP, ONE INTERNATIONAL PLACE, BOSTON, MA, 02110, US

(21) Appl. No.: 12/024,866

(22) Filed: Feb. 1, 2008

Related U.S. Application Data
(60) Provisional application No. 60/899,117, filed on Feb. 2, 2007.

(51) Int. Cl. A61B 18/18

(52) U.S. Cl. 606/9

(57) ABSTRACT

A handpiece for a dermatologic treatment includes a cooling fluid module and a gas source. The cooling fluid module contains cooling fluid in a liquid state. The gas source provides a flow of gas to cool the handpiece to maintain the cooling fluid in the liquid state prior to delivery of cooling fluid spray to a target region of skin.

### CROSS-REFERENCE TO RELATED APPLICATION

This application claims the benefit of and priority to U.S. Provisional Application Ser. No. 60/899,117, filed Feb. 2, 2007, which is owned by the assignee of the instant application and the disclosure of which is incorporated herein by reference in its entirety.

### FIELD OF THE INVENTION

The invention relates generally to handpieces used for cosmetic and/or dermatologic treatment, and more particularly, to using a flow of gas to maintain cooling fluid in a liquid state in a handpiece prior to delivery of cooling fluid spray to a target region of skin.

### BACKGROUND OF THE INVENTION

Cosmetic and/or dermatologic treatments can be performed by delivering radiation non-invasively to target regions of skin. Radiation can be provided by radiation sources such as lasers and/or pulsed light sources. However, the delivery of radiation can cause a recipient some discomfort, and a treatment can include cooling to protect the skin surface, to minimize unwanted injury to the surface of the skin, and to minimize any pain that a patient may feel.

A handpiece for use in the cosmetic or dermatologic treatment can contain a cooling fluid. An operator of the handpiece can deliver a spray of cooling fluid to a target region of skin prior to, during, and/or after delivery of radiation. Delivery of the radiation can heat the handpiece, which can cause at least some of the cooling fluid to vaporize. An operator may need to halt a treatment until the handpiece cools sufficiently and the cooling fluid returns to the liquid state. Furthermore, the handpiece can become uncomfortable for the operator to hold.

In addition, debris can build up on optical components used to deliver the radiation. The debris can include skin tissue, vapor, smoke, and/or liquid used to cool the skin surface. Debris can contaminate an optical component or accumulate in the optical path, resulting in a loss of transmission of light and/or damage to an optical component. The operator may need to halt treatment periodically to wipe the optical component.

### SUMMARY OF THE INVENTION

In various embodiments, the invention features a handpiece that can maintain a cooling fluid in a liquid state during a cosmetic and/or dermatologic treatment. A flow of gas can be used to maintain the cooling fluid in the liquid state or cause the cooling fluid to liquefy. The flow of gas can prevent debris from contacting an optical component of the handpiece or from accumulating in the optical path of radiation.

In one aspect, there is a handpiece for a dermatologic treatment. The handpiece includes a cooling fluid module and a gas source. The cooling fluid module contains cooling fluid in a liquid state. The gas source provides a flow of gas to cool the handpiece to maintain the cooling fluid in the liquid state prior to delivery of cooling fluid spray to a target region of skin.

In another aspect, there is a method including containing a cooling fluid in a cooling fluid module of a handpiece for a dermatologic treatment and flowing a gas to maintain the cooling fluid in a liquid state prior to delivery of cooling fluid spray to a target region of skin.

In another aspect, there is a dermatologic treatment apparatus including a main unit, a delivery apparatus, and a handpiece. The main unit includes a cooling fluid source, a gas source, and a radiation source. The delivery apparatus is coupled to the main unit. The delivery apparatus includes a first conduit that receives cooling fluid from the cooling fluid source, a second conduit that receives gas from the gas source, and a third conduit that receives radiation from the radiation source. The handpiece includes a cooling fluid module containing cooling fluid received from the first conduit. The handpiece receives a flow of gas from the second conduit. The flow of gas maintains the cooling fluid in a liquid state prior to delivery of cooling fluid spray to a target region of skin.

In other examples, any of the aspects above, or any apparatus or method described herein, can include one or more of the following features. The handpiece can include a sensor to monitor the cooling fluid module to determine the state of matter of the cooling fluid. The sensor can monitor the optical transmission through the cooling fluid module. The sensor can monitor the pressure of the cooling fluid module. The handpiece can be capable of delivering the cooling fluid spray when the sensor determines that the cooling fluid is in the liquid state. The flow of gas from the gas source can maintain the temperature of the handpiece below about 100° F. The rate of the flow of gas can be about 40 L/min. The handpiece can include at least one optical component for directing radiation from a radiation source to a target region of skin through an optical path. The flow of gas can cool the at least one optical component. The flow of gas can prevent debris from contacting the at least one optical component or from accumulating in the optical path of radiation. The main unit can include a gas blower and/or a heat exchanger.

Other aspects and advantages of the invention will become apparent from the following drawings and description, all of which illustrate the principles of the invention, by way of example only.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead being placed upon illustrating the principles of the invention.

FIG. 1 is an exemplary embodiment of a system for dermatologic treatments.

FIG. 2 is an exemplary embodiment of a system for dermatologic treatments.

FIG. 3 is an exemplary embodiment of a handpiece for use in systems for dermatologic treatments.

FIG. 4 is an exemplary embodiment of a system for dermatologic treatments.

FIG. 5 is an exemplary embodiment of a handpiece for use in systems for dermatologic treatments.

### DESCRIPTION OF THE INVENTION

Various skin conditions can be treated by delivering radiation non-invasively to target regions of skin. A handpiece used in the treatment can reduce the discomfort caused by the radiation by delivering a cooling fluid to cool the target region. During a treatment, delivery of radiation can heat the handpiece, vaporizing at least part of the cooling fluid. Delivery of radiation can also create debris. A flow of gas can cool the handpiece, maintain the cooling fluid in a liquid state, and prevent debris from contacting the handpiece or accumulating in the path of radiation.

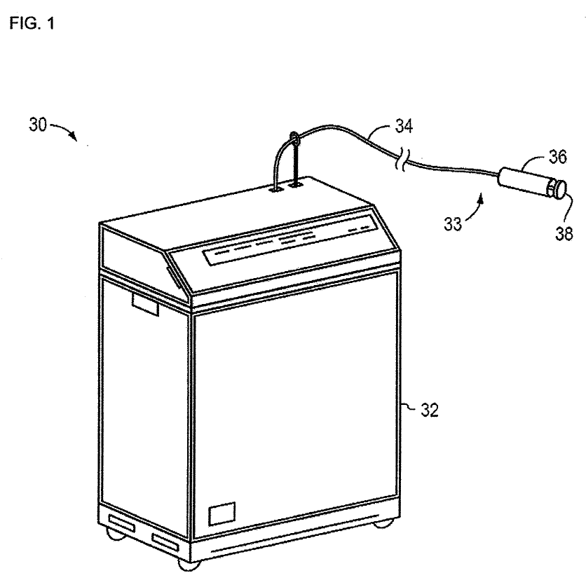

FIG. 1 shows an exemplary embodiment of a system 30 for dermatologic treatments. The system 30 can be used to deliver non-invasively a beam of radiation to a target region. For example, the beam of radiation can be delivered through an external surface of skin over the target region. The system 30 includes a main unit 32 and a delivery system 33. The main unit 32 can include a radiation source that generates a beam of radiation. In one embodiment, the beam of radiation provided by the main unit 32 is directed via the delivery system 33 to a target region. The delivery system 33 can include a umbilicus 34 having a substantially circular cross-section and a handpiece 36. The beam of radiation can be delivered by an optical fiber of the umbilicus 34 to the handpiece 36, which can include an optical system (e.g., an optic or system of optics) to direct the beam of radiation to the target region. A user can hold or manipulate the handpiece 36 to irradiate the target region. The delivery system 33 can be positioned in contact with a skin surface, can be positioned adjacent a skin surface, can be positioned proximate a skin surface, can be positioned spaced from a skin surface, or a combination of the aforementioned. In the embodiment shown, the delivery system 33 includes a spacer 38 to space the delivery system 33 from the skin surface. In one embodiment, the spacer 38 can be a distance gauge, which can aid a practitioner with placement of the delivery system 33.

To minimize unwanted thermal injury to tissue not targeted (e.g., an exposed surface of the target region and/or the epidermal layer), the system 30 can include a cooling system for cooling before, during or after delivery of radiation, or a combination of the aforementioned. Cooling can include contact conduction cooling, evaporative spray cooling, convective air flow cooling, or a combination of the aforementioned.

In one embodiment, the handpiece 36 can include a skin contacting portion that can be brought into contact with the skin. The skin contacting portion can include a sapphire or glass window and a fluid passage containing a cooling fluid. The cooling fluid can be a fluorocarbon type cooling fluid, which can be transparent to the radiation used. The cooling fluid can circulate through the fluid passage and past the window to cool the skin.

In another embodiment, the handpiece 36 can include a spray cooling device that uses a coolant, such as cryogen, water, and/or air. The coolant can be a liquid form of any gas, such as carbon dioxide. In one embodiment, a dynamic cooling device can be used to cool the skin (e.g., a DCD available from Candela Corporation). For example, the umbilicus 34 can include a conduit, such as tubing, for delivering a cooling fluid to the handpiece 36. The conduit can be connected to a container of a low boiling point fluid located in the main unit 32, and the handpiece can include a valve for delivering a spurt of the fluid to the targeted region of skin. Heat can be extracted from the skin by the evaporative cooling of the low boiling point fluid. The fluid can be a non-toxic substance with high vapor pressure at normal body temperature, such as a cryogenic fluid, Freon, tetrafluoroethane, or liquefied CO₂.

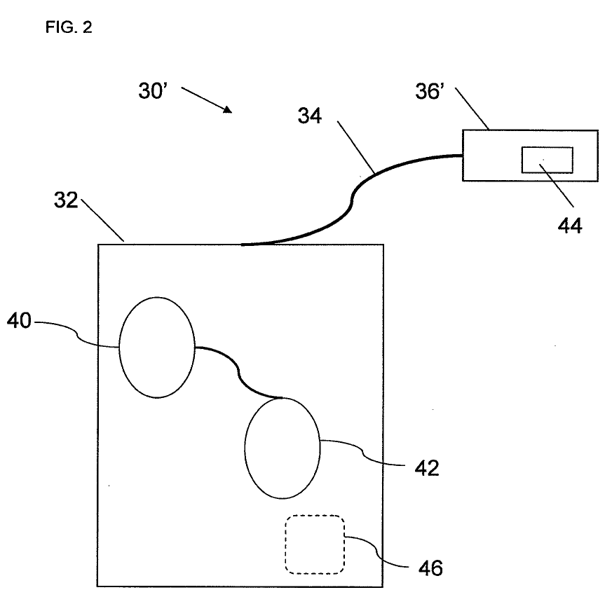

FIG. 2 shows another exemplary embodiment of a system 30′ for dermatologic treatments. The system 30′ including a main unit 32 and an umbilicus 34 connecting the main unit 32 to a handpiece 36′. The main unit 32′ can include a user interface 40 and a processing unit 42. The umbilicus 34 can include one or more conduits for communicating power, signal, fluid, and/or gas between the main unit 32 and the handpiece 36′. The handpiece 36′ can include a radiation module 44, such as a diode laser. The handpiece 36′ can include other components, such as filters and/or optics for delivering the radiation to biological tissue. Power from the main unit 32 can be used to drive the radiation module 44, and signal from the main unit 32 can be used to control the output of the radiation module 44 (e.g., set, maintain, or control parameters of radiation being emitted from the radiation module 44). The fluid and/or gas can be used to cool the radiation module 44 and/or a transparent or translucent member contacting the skin during treatment. The main unit 32 can include a memory module 46.

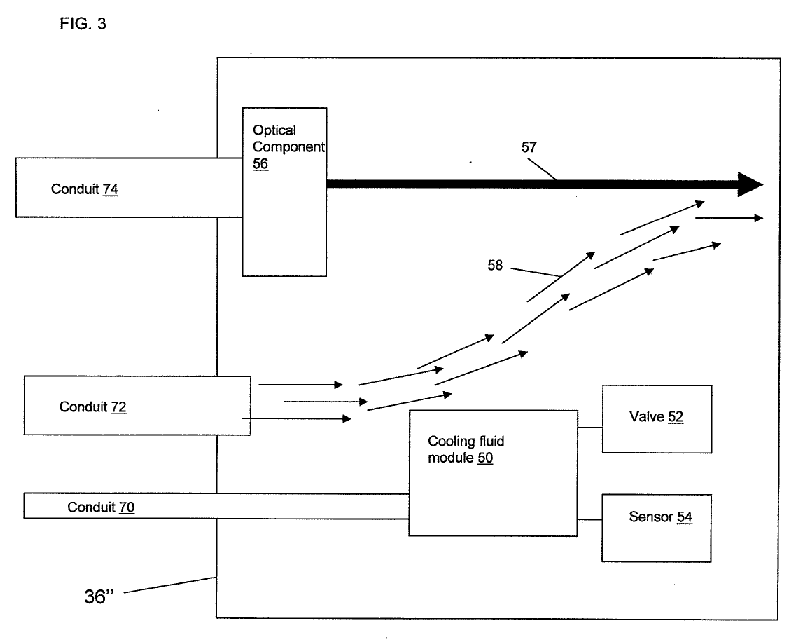

FIG. 3 shows an exemplary embodiment of a handpiece 36″ for use in systems for dermatologic treatments. The handpiece 36″ can include a cooling fluid module 50, a valve 52, and a sensor 54 coupled to the cooling fluid module 50. The handpiece 36″ can include at least one optical component 56, which can receive radiation and deliver the radiation to the target region of skin. The handpiece 36″ can be connected to conduits 70, 72, and 74.

Conduit 72 can deliver a flow of gas (generally shown by the arrows 58). Conduit 70 can deliver cooling fluid to the cooling fluid module 50, which can contain cooling fluid in a liquid state. The cooling fluid module 50 can be a reservoir for containing cooling fluid, a portion of the conduit 70, valve 52 (or a portion thereof), or a combination of the aforementioned.

The sensor 54 can monitor at least one characteristic of the cooling fluid in the cooling fluid module 50. The valve 52 can deliver a spray of cooling fluid from the cooling fluid module 50 to a target region, such as an external surface of skin. The at least one optical component 56 can receive a beam of radiation from conduit 74. The handpiece 36″ can receive the flow of gas from a gas source external to the handpiece 36″, e.g., via conduit 72.

During a dermatologic treatment, an operator uses the handpiece 36″ to deliver the beam of radiation to a target region. To minimize unwanted thermal injury to tissue not targeted, a spray of cooling fluid can be delivered to the target region before, during, or after delivery of radiation, or a combination of the aforementioned. Heat can be extracted from the skin by the evaporative cooling of the cooling fluid, which can have a low boiling point.

The radiation can heat the handpiece 36″. If the handpiece 36″ reaches a temperature higher than the temperature of the cooling fluid, the cooling fluid begins to vaporize. The valve 52 then delivers a mixture of liquid and gaseous cooling fluid to the target region. If enough cooling fluid vaporizes, the valve 52 can be unable to deliver the liquid spray of cooling fluid at all. Thus, the handpiece's 36″ ability to cool the skin diminishes. The handpiece 36″ can receive a flow of gas to cool the handpiece 36″ and maintain the cooling fluid in a liquid state and/or reliquify vaporous cooling fluid. The rate of flow can be, for example, about 40 liters/minute. Advantageously, the flow of gas can prevent or reduce the likelihood of cooling fluid vaporization.

The sensor 54 can monitor at least one characteristic of the cooling fluid in the cooling fluid module 50. If the characteristic indicates that the cooling fluid is vaporizing, the handpiece 36″ can enter a non-operational state. For example, the sensor 54 can monitor optical transmission through any part of the cooling fluid module 50. If the optical transmission detects gas in the monitored part of the cooling fluid module 50, the handpiece 36″ can halt delivery of the radiation and enter a standby state. In some embodiments, the sensor 54 can monitor the pressure or the partial pressure in the cooling fluid module 50. If the pressure is below a desired threshold, the handpiece 36″ can enter the standby state. For example, the handpiece 36″ can enter the standby state if the sensor 54 senses a pressure below about 115-118 psig. Advantageously, the handpiece 36″ can halt operation when the cooling fluid includes a predetermined determined amount of vapor and the ability to cool the skin is diminished.

The handpiece 36″ can exit the non-operational state, for example, when the operator pushes a button to resume treatment. In the non-operational state, the sensor 54 can continue to monitor at least one characteristic of the cooling fluid in the cooling fluid module 50. Optionally, the handpiece 36″ can be non-responsive to an attempt to resume treatment if the monitored characteristic continues to indicate that the cooling fluid includes the predetermined amount of vapor.

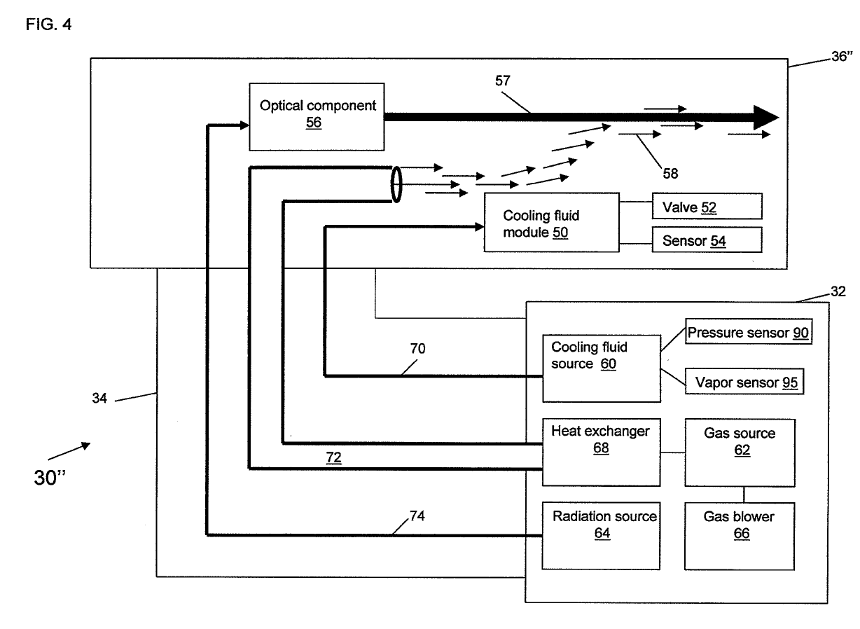

FIG. 4 shows an exemplary embodiment of a system 30″ for dermatologic treatments. The system 30″ can include a main unit 32 and an umbilicus 34 connecting the main unit 32 to a handpiece 36″. The main unit 32 can include a cooling fluid source 60, a gas source 62, and a radiation source 64. The main unit 32 can include a pressure sensor 90 and a vapor sensor 95, both coupled to the cooling fluid source 60. The main unit 32 can include a gas blower 66 and a heat exchanger 68. Alternatively, the gas blower 66 can be a separate add-on attachment to the handpiece 36″ or the main unit 32. The umbilicus 34 can include a first conduit 70 connected to the cooling fluid source 60, a second conduit 72 connected to the gas source 62 or the gas blower 66, and/or a third conduit 74 connected to the radiation source 64. The conduits can be, for example, optical fibers and/or tubes (e.g. Teflon tubes). The umbilicus 34 can include conduits in addition to conduits 70, 72, and 74. The handpiece 36″ can include a cooling fluid module 50 connected to the first conduit 70 of the umbilicus 34, a valve 52, a sensor 54 coupled to the cooling fluid module 50, at least one optical component 56, a flow of gas 58 from the second conduit 72 of the umbilicus 34, and a beam of radiation 57 from the third conduit 74 of the umbilicus 34. In some embodiments, the handpiece 36″ can contain a radiation source, such as a diode laser, to produce the beam of radiation.

During a treatment, an operator can use the handpiece 36″ to deliver radiation to a target region of skin on a patient. The radiation can originate in the radiation source 64 of the main unit 32. The third conduit 74 in the umbilicus 34 can transmit a beam of radiation from the radiation source 64 to the handpiece 36″. The operator can use the handpiece 36″ to control the beam of radiation delivered to the target region. The optical component 56 can receive the beam of radiation from the third conduit 74 and direct the beam to the target region, thereby defining an optical path.

Applying a cooling fluid to the target region can increase the comfort of the patient and minimize thermal injury to untargeted regions. The valve 52 can deliver a spray of cooling fluid from the cooling fluid module 50 before, during, and/or after the delivery of radiation to the target region. As treatment progresses, the cooling fluid source 60 can provide additional cooling fluid through the first conduit 70 in the umbilicus 34. Pressure in the cooling fluid source 60 can propel the additional cooling fluid to the handpiece 36″. The cooling fluid source 60 can be heated to maintain a desired temperature or pressure within the source 60. The desired temperature can be about 100° F., and the desired pressure can be about 120 psig.

The flow of gas 58 from the second conduit 72 can cool the handpiece 36″ and maintain the cooling fluid in the liquid state and/or reliquify vaporous cooling fluid. The flow of gas can originate in the gas source 62 in the main unit 32. The gas source 62 can be a canister of gas or ambient room air. The gas blower 66 can move the gas from the gas source 62 through the second conduit 72 (for example, an 8 mm ID polymer tube) to the handpiece 36″. As the gas blower 66 and/or other components of the main unit 32 (e.g. radiation source 64) can heat the gas, the gas can pass through the heat exchanger 68 to be cooled. The heat exchanger 68 can be made of copper.

During a treatment, the pressure sensor 90 can monitor the pressure of the cooling fluid source 60. If the pressure in the cooling fluid source 60 is too low, the cooling fluid can vaporize. A low pressure can indicate a low level of cooling fluid in the cooling fluid source source 60. If the pressure sensor 90 detects a pressure below a desired threshold (e.g., about 115-118 psig), the handpiece 36″ can enter a non-operational state and halt operation of the radiation. The main unit 32 can further heat the cooling fluid source 60 to increase the pressure.

The vapor sensor 95 can monitor the cooling fluid source 60 for a bubble. If the vapor sensor 95 detects a bubble, the cooling fluid in the cooling fluid source 60 is low and needs to be refilled or replaced. The main unit 32 can force the handpiece 36″ into a non-operational state until the cooling fluid source 60 is replaced or replenished.

The handpiece 36″ can exit the non-operational state, for example, when the operator pushes a button to resume treatment. In the non-operational state, any of the sensor 54, pressure sensor 90, or vapor sensor 95 can continue monitoring their respective characteristics. Optionally, the handpiece 36″ can be non-responsive to any attempt to resume treatment if any one of the monitored characteristics continues to indicate that the cooling fluid is vaporizing in part or the cooling fluid source is low.

As the treatment progresses, the repeated delivery of radiation can produce debris, such as smoke, vapor, and/or dermatologic tissue. The flow of gas through the handpiece 36″ can prevent debris from accumulating on the optical component or in the optical path of radiation. The handpiece 36″ can be internally sealed except for a hole positioned after the last optical component directing radiation to the target region of skin. The flow of gas can exit the handpiece 36″ at the hole and enter the optical path. The gas can traverse the optical path out the handpiece, providing a positive pressure to prevent ejected debris off the skin from getting on or into the handpiece or accumulating in the optical path. The gas flow can be about 40 liters per minute, although faster or slower flow can be used depending on the application.

The gas can enter the optical path after the last optical component. In certain embodiments, the gas can enter the optical path before the last optical component of the handpiece 36″ and exit the handpiece 36″ though an aperture in the last optical component. In some embodiments, the gas can exit the handpiece 36″ through an aperture in the housing of the handpiece 36″ positioned substantially at the last optical component.

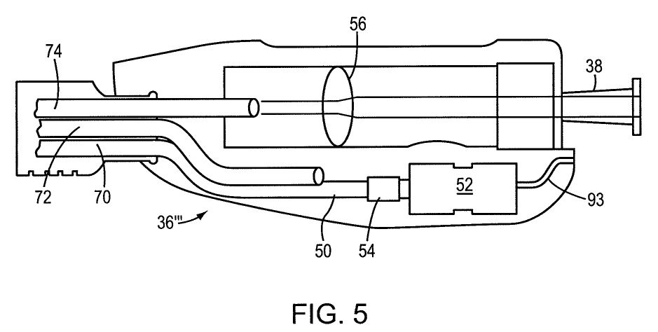

FIG. 5 shows an exemplary embodiment of a handpiece 36″′ for use in a cosmetic and/or dermatologic treatment. The handpiece 36″′ can include a cooling fluid module 50, a valve 52, a nozzle 93, a sensor 54, and an optical component 56. The handpiece can include a conduit 74 for delivering a beam of radiation from a radiation source external to the handpiece. The handpiece 36″′ can include a spacer 38 to space the handpiece 36″′ from the skin surface. The handpiece 36″′ can be connected to a conduit 72 for delivering a flow of gas to cool the handpiece 36″′ and maintain the cooling fluid in the liquid state. The flow of gas can enter the beam path and provide a positive pressure for keeping debris from entering the handpiece 36″′ or accumulating in the optical path.

In various embodiments, the radiation source in the main unit 32 can be an incoherent light source, a coherent light source (e.g., a laser), a microwave generator, or a radio-frequency generator. In one embodiment, the source generates ultrasonic energy that is used to treat the tissue. In some embodiments, two or more sources can be used together to effect a treatment. For example, an incoherent source can be used to provide a first beam of radiation while a coherent source provides a second beam of radiation. The first and second beams of radiation can share a common wavelength or can have different wavelengths. In an embodiment using an incoherent light source or a coherent light source, the beam of radiation can be a pulsed beam, a scanned beam, or a gated continuous wave (CW) beam. In some embodiments, two lasers can be used (e.g., a 755 nm alexandrite laser and a 1064 nm Nd:YAG laser). Exemplary commercial laser sources include, but are not limited to, GENTLELASE, GENTLEYAG and GENTLEMAX available from Candela Corporation (Wayland, Mass.).

In various embodiments, the system 30′, 30′, or 30″ can be a fluorescent pulsed light (FPL) or an intense pulsed light (IPL) system. FPL technologies can utilize laser-dye impregnated polymer filters to convert unwanted energy from a xenon flashlamp into wavelengths that enhance the effectiveness of the intended applications. FPL technologies can be more energy efficient and can generate significantly less heat than comparative IPL systems. A FPL system can be adapted to operate as a multi-purpose treatment system by changing filters or handpieces to perform different procedures. For example, separate handpieces allow a practitioner to perform tattoo removal and other vascular treatments.

In various embodiments, the beam of radiation can have a wavelength between about 380 nm and about 2,600 nm, although longer and shorter wavelengths can be used depending on the application. In some embodiments, the wavelength can be between about 1,000 nm and about 2,200 nm. In other embodiments, the wavelength can be between about 1,160 nm and about 1,800 nm. In yet other embodiments, the wavelength can be between about 1,190 nm and about 1,230 nm or between about 1,700 nm and about 1,760 nm. In one embodiment, the wavelength is about 1,210 nm or about 1,720 nm. In one detailed embodiment, the wavelength is about 1,208 nm, 1,270 nm, 1,310 nm, 1,450 nm, 1,550 nm, 1,720 nm, 1,930 nm, or 2,100 nm. One or more of the wavelengths used can be within a range of wavelengths that can be transmitted to fatty tissue and absorbed by the fatty tissue in the target region of skin.

In various embodiments, the beam of radiation can have a fluence between about 0.1 J/cm² and about 600 J/cm², although higher and lower fluences can be used depending on the application. In some embodiments, the fluence can be between about 10 J/cm² and about 150 J/cm². In one embodiment, the fluence is between about 5 J/cm² and about 100 J/cm².

In various embodiments, the beam of radiation can have a spotsize between about 0.1 mm and about 30 mm, although larger and smaller spotsizes can be used depending on the application.

In various embodiments, the beam of radiation can have a pulse duration between about 10 μs and about 30 s, although larger and smaller pulse durations can be used depending on the application. In one embodiment, the beam of radiation can have a pulse duration between about 0.1 second and about 20 seconds. In one embodiment, the beam of radiation can have a pulse duration between about 1 second and 20 seconds. In certain embodiments, the beam of radiation can be delivered in a series of sub-pulses spaced in time such that within a region of tissue, the tissue is exposed to radiation intermittently over total time interval of between about 0.1 second and about 20 seconds.

In various embodiments, the beam of radiation can be delivered at a rate of between about 0.1 pulse per second and about 10 pulses per second, although faster and slower pulse rates can be used depending on the application.

In various embodiments, the parameters of the radiation can be selected to deliver the beam of radiation to a predetermined depth. In some embodiments, the beam of radiation can be delivered to the target region about 0.5 mm to about 10 mm below an exposed surface of the skin, although shallower or deeper depths can be selected depending on the application. In one embodiment, the beam of radiation is delivered to the target region about 1 mm to about 10 mm below an exposed surface of the skin.

In various embodiments, the tissue can be heated to a temperature of between about 50° C. and about 80° C., although higher and lower temperatures can be used depending on the application. In one embodiment, the temperature is between about 55° C. and about 70° C.

Skin conditions that can be treated include, but are not limited to, vascular lesions, hirsutism, port wine stains, hemangiomas, telangiectasis, angiomas, adenoma sebaceum, angiokeratomas, venous lakes, spider veins, rosacea, poikloderma of civatte, pigmented lesions, cellulite, fatty tissue, lentigo, nevus of ota, nevus of ito, blue nevus, ephelides, becker's nevi, hairy nevi, epidermal, melanosis, nevus spilus, hyper-pigmentation, skin cancers (e.g., with PDT), acne vulgaris, acne scars, hypertrophic scars, rhytides, hypertrichosis, hidradenitis, suppurative, pseudo-folliculitis, barbae, tattoos, chrysiasis, excessive or unwanted hair, and adipose contouring, removal, and/or reduction.

While the invention has been particularly shown and described with reference to specific illustrative embodiments, it should be understood that various changes in form and detail may be made without departing from the spirit and scope of the invention.

1. A handpiece for a dermatologic treatment, comprising:
a cooling fluid module containing cooling fluid in a liquid state; and
a gas source providing a flow of gas to cool the handpiece to maintain the cooling fluid in the liquid state prior to delivery of cooling fluid spray to a target region of skin.

2. The handpiece of claim 1 further comprising a sensor to monitor the cooling fluid module.

3. The handpiece of claim 2 wherein the sensor monitors the optical transmission through the cooling fluid module to determine the state of matter of the cooling fluid.

4. The handpiece of claim 2 wherein the handpiece is capable of delivering the cooling fluid spray when the sensor determines that the cooling fluid is in the liquid state.

5. The handpiece of claim 1 wherein the flow of gas from the gas source maintains the temperature of the handpiece below about 100° F.

6. The handpiece of claim 1 wherein the rate of the flow of gas is about 40 L/min.

7. The handpiece of claim 1 further comprising at least one optical component for directing radiation from a radiation source to the target region of skin through an optical path, the flow of gas cooling the at least one optical component.

8. The handpiece of claim 7 wherein the flow of gas prevents debris from contacting the at least one optical component or from accumulating in the optical path of radiation.

9. A method comprising:
containing cooling fluid in a cooling fluid module of a handpiece for a dermatologic treatment; and
flowing a gas to maintain the cooling fluid in a liquid state prior to delivery of cooling fluid spray to a target region of skin.

10. The method of claim 9 wherein the rate of the flow of gas is about 40 L/min.

11. The method of claim 9 further comprising enabling the handpiece to deliver the cooling fluid spray when the sensor determines that the cooling fluid is in the liquid state.

12. The method of claim 9 further comprising monitoring the optical transmission through the cooling fluid module.

13. A dermatologic treatment apparatus comprising:
a main unit comprising a cooling fluid source, a gas source, and a radiation source;
a delivery apparatus coupled to the main unit, the delivery apparatus comprising a first conduit that receives cooling fluid from the cooling fluid source, a second conduit that receives gas from the gas source, and a third conduit that receives radiation from the radiation source; and
a handpiece comprising a cooling fluid module containing cooling fluid received from the first conduit, the flow of gas received from the second conduit maintaining the cooling fluid in a liquid state prior to delivery of cooling fluid spray to a target region of skin.

14. The apparatus of claim 13 wherein the handpiece further comprises at least one optical component for directing radiation from the third conduit to the target region of skin through an optical path, the flow of gas cooling the at least one optical component.

15. The apparatus of claim 13 wherein the flow of gas enters the optical path of the radiation from the third conduit to prevent debris from contacting the at least one optical component or from accumulating in the optical path of radiation.

16. The apparatus of claim 13 wherein the main unit further comprises a gas blower for moving the gas in the gas source to the second conduit.

17. The apparatus of claim 13 wherein the main unit further comprises a heat exchanger for cooling the gas from the gas source.

18. The apparatus of claim 13 wherein the main unit further comprises a pressure sensor for monitoring the pressure of the cooling fluid source.

19. The apparatus of claim 13 wherein the main unit further comprises a vapor sensor for monitoring the cooling fluid source for bubbles.
